(19) United States
(12) Patent Application Publication
(10) Pub. No.: US 2008/0125870 A1
(43) Pub. Date: May 29, 2008

(54) NERVE REGENERATION DEVICE

(76) Inventors: Ralph W. Carmichael, Phoenix, AZ (US); Bradley C. Fox, Phoenix, AZ (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 11/936,078

(22) Filed: Nov. 6, 2007

Related U.S. Application Data
(60) Provisional application No. 60/857,233, filed on Nov. 6, 2006.

(51) Int. Cl. A61F 2/02; G03F 7/00; C08F 18/02; A61N 1/30

(52) U.S. Cl. 623/23.72; 430/322; 526/292.6; 29/432.2; 29/428; 604/19

(57) ABSTRACT

Devices for use in the regeneration or repair of body tissue (such as nerves) comprise a multi-lumen scaffold and, optionally, an outer sheath. The tissue guidance conduits are preferably formed of biocompatible, biodegradable charged polymer hydrogels, particularly charged oligo-(polyethylene glycol)fumarate hydrogels. The outer sheath is formed of a stronger material than the scaffold and preferably comprises a region at each end for suturing the device in place. Methods for making tissue guidance conduits and for repairing tissue are also described.

### CROSS-REFERENCE TO RELATED APPLICATION

This application claims priority to U.S. Provisional Patent Application Ser. No. 60/857,233, filed Nov. 6, 2006, the content of which is incorporated herein by reference.

### FIELD OF THE INVENTION

This invention relates generally to the field of medical devices, particularly implantable tissue regeneration devices for use in the regeneration or repair of body tissues, particularly nerves. In particular, the present invention relates to tissue guidance conduits comprising at least one, preferably multi-lumen, scaffold and, optionally, at least one outer sheath.

This invention also pertains to biocompatible, biodegradable charged hydrogel polymers, particularly positively charged hydrogel polymers, and to the formation of tissue guidance conduits from the same, wherein the scaffold of the tissue guidance conduit comprises a charged polymer hydrogel. Other aspects of the invention relate to methods for making tissue guidance conduits and for using same in nerve regeneration, bone, soft tissue, muscle and cartilage repair and regeneration, treatment of burns, DNA delivery, and cell transplantation.

### BACKGROUND OF THE INVENTION

When nerves are severed due to accidental trauma or surgery direct coaptation of the severed nerve ends provides the best opportunities for nerve regeneration. In cases where it is not possible to directly join the ends a “nerve gap” exists and a bridge must be provided between the severed ends to assist nerve regeneration. One method to bridge a nerve gap is to use another nerve, such as the sural nerve. However, this method has the disadvantage that there is only a limited amount of nerve tissue available from this source (both in length and diameter), and removal of the sacrificial nerve results in an area of numbness at the donor site and other side effects, such as, scaring and pain for the patient.

An alternative method to bridge a nerve gap is to use a synthetic nerve tube or nerve guide as a conduit for nerve axon regeneration. Single lumen tubes have been described for nerve axon growth, such as, for example, those described in U.S. Pat. Nos. 4,870,966 and 5,147,399 to Dellon et al. One disadvantage of single lumen tubes is that axons regenerating across single tubes or guides may disperse resulting in inappropriate target reinnervation. This can produce the undesired results of co-contraction of opposing muscles or synkinesis in a patient.

Existing methods for the construction of nerve guidance conduits primarily use molding techniques, such as injection molding. Channels are introduced, for example, using wires or fibers within the mold, such as set forth in U.S. Pat. Nos. 6,214,021 to Hadlock et al. and 6,090,117 and 6,589,257 to Shuimizu. Extrusion methods may also be used. Such methods have the disadvantage that it is difficult to form man-made structures to conform to nature's tissue designs (such structures that do relatively conform are referred to herein as “biomimetic”).

There is a continuing need for improved materials, designs and methods for synthetic tissue guidance conduits (particularly for nerves), and for other tissue generation purposes that may provide one or more of the following benefits, depending upon the application: (1) a synthetic, multi-channel tissue guidance conduit for axon regeneration that limits axonal dispersion and promotes axon growth, (2) a tissue guidance conduit that is able to separately guide groups of regenerating axons, and which is constructed of a material that is biocompatible, and (3) a tissue guidance conduit that bio-mimics the biological architecture of the tissue to be repaired.

The present invention addresses these needs, as well as others, by providing tissue guidance conduits comprising biocompatible polymers such as charged hydrogels, and methods of making the same, for the regeneration and repair of nerve defects and other applications.

As used herein, the following terms have the meanings ascribed below:
(1) “Scaffold” means a structure that is part of a tissue guidance conduit and that has one or more lumens.
(2) “Lumen” means a passage in a scaffold or other structure.
(3) “Tissue regeneration device,” “tissue guidance conduit,” or “guidance conduit” means a device according to the invention that includes at least one scaffold and optionally other structures, such as other scaffolds or one or more outer sheaths.
(4) “Outer sheath” means an outer cover on at least a portion of scaffold that is preferably comprised of a material different than the material comprising the scaffold.
(5) “Biocompatible material” is a material that stimulates only a mild, often transient, implantation response, as opposed to a severe or escalating response in a patient.
(6) “Biodegradable material” refers to a material that under normal in vivo physiological conditions is capable of being degraded by biological processes into components that can be metabolized and/or excreted from a patient.
(7) “Bioresorbable” or “bioabsorbable” refers to a material that breaks down over a period of time due to the chemical/biological actions of the body.
(8) “Defect” as used in connection with a tissue of the body means a cut, tear, break or other defect that can potentially be repaired utilizing a tissue guidance conduit or other structure using a charged hydrogel configured to repair the defect.

### SUMMARY OF THE INVENTION

One aspect of the present invention relates to the design and manufacture of scaffolds or other structures from charged hydrogel polymers (preferably positively charged) and their use for tissue engineering, nerve regeneration, bone, soft tissue, muscle and cartilage repair and regeneration, treatment of burns, DNA delivery and cell transplantation purposes.

The tissue regeneration devices of the present invention preferably comprise at least one scaffold and one or more lumens running through each scaffold from the first end to the second end of the scaffold. The tissue regeneration devices optionally comprise an outer sheath formed of a material with stronger material properties than the scaffold(s) so the outer sheath can be sutured securely to tissue (such as nerve ends) without tearing.

In one embodiment, a scaffold is formed, an outer sheath is formed, and at least a portion of the scaffold is then inserted into the outer sheath.

The tissue guidance conduit may be constructed so as to mimic the biological architecture of the tissue, such as a nerve, that is to be repaired. This may include, but is not limited to, one or more of splitting the lumen into separate lumens to mimic the design of nerve bundles, choosing the dimensions of the lumen(s) and the tissue guidance conduit to match a tissue, having tissue guidance conduits with an oval cross section or other cross section to match the application, furcation of the tissue guidance conduit into branching trunks, and/or changing the cross-section of one or more lumens along the length of the device. In one embodiment, the tissue guidance conduit of the present invention comprises a multi-lumen scaffold and an outer sheath, similar to the structure of nerves.

A tissue guidance conduit of the present invention provides, when used with nerves, the advantage of limiting axon dispersion, leading to improved nerve regeneration and repair by separately guiding groups of regenerating axons. Generally, these tissue guidance conduits promote better axon growth and improve the targeting of regenerating axons to the correct muscle.

Some embodiments of the present invention are directed to methods of using particularly-configured, charged hydrogel polymers to repair body tissues. A charged substrate including a charged hydrogel polymer is then applied to a region of body tissue, nerve, bone, cartilage, muscle or other tissue to be repaired. The charged hydrogel polymer stimulates cellular growth thereby promoting repair and/or healing of the body tissue. In this embodiment, in addition to forming the charged hydrogel as a scaffold, it may be formed as a sheet (for example, to apply to a burned area) or a tape (for example, to wrap around a torn tendon or broken bone), or any other suitable shape to be applied to a tissue to be repaired. The hydrogel may also be applied as a liquid or near liquid (hereafter, “liquid”) and be cured in-situ in order to conform to the tissue area to be repaired.

Any of the embodiments of the invention may provide for the controlled delivery of a bioactive agent to a target site utilizing a charged hydrogel, either in the form of a scaffold or otherwise. The method may include forming a charged substrate that includes a charged hydrogel polymer, incorporating a bioactive agent into the substrate, and placing the substrate at a target site. In some embodiments, the bioactive agent may be one or more of a gene, drug, or cells.

### BRIEF DESCRIPTION OF THE DRAWINGS

FIGS. 1(a) and (b) show side and end cross-sections of one embodiment of the nerve guidance conduits of the present invention comprising an inner scaffold and an outer sheath.

FIG. 2 shows a scaffold with lumen bundles A and B separated by a septum according to the present invention.

FIG. 3 shows a side view and an end view of one embodiment of a scaffold according to the invention.

FIGS. 4(a) to (e) show examples of branching scaffold embodiments according to the invention.

FIG. 5 shows an example of branching scaffold embodiments according to the invention.

FIG. 6 shows an embodiment of the present invention where an outer sheath surrounds a portion of the outer surface of each end of the scaffold.

FIG. 7 shows ATR-FTIR spectra of crosslinked hydrogels with and without MAETAC.

FIG. 8 shows solid C¹³ NMR spectra of crosslinked hydrogels with and without MAETAC.

FIG. 9 shows swelling of positively charged hydrogels in PBS and water.

FIG. 10 shows the compressive modulus of OPF hydrogels as a function of the percentage of MAETAC.

FIG. 11 shows cell viability as a function of the percentage of MAETAC.

FIG. 12 shows MSCs attached to (a) unmodified, and (b) charge modified hydrogels (0.2 M MAETAC).

### DETAILED DESCRIPTION

The present invention pertains generally to medical devices and, in particular, to implantable tissue guidance conduits for the regeneration and/or self-repair of nerve defects in patients with injured, severed or potentially otherwise defective tissue (particularly nerves) suitable for repair utilizing the invention. The devices are particularly useful for the repair and regeneration of peripheral nerves.

A tissue guidance conduit of the present invention comprises at least one scaffold through which runs one or more lumens. The scaffold is formed of a soft material, preferably a polymer that is biocompatible, such as a hydrogel synthesized to hold a charge as described herein. The scaffold may also further comprise one or more bio-active agents, such as nerve growth factors, that promote nerve regeneration (if being used to regenerate nerve tissue) or other bio-active agents.

The scaffold(s) is optionally surrounded by an outer sheath. The outer sheath is formed of a material with mechanical properties sufficient to allow it to be securely sutured in place, whereas the material of the scaffold may be too soft to be securely sutured without the support of the outer sheath. The outer sheath may also incorporate perforations to enhance mass transport therethrough.

The tissue guidance conduits of the present invention are biocompatible, and are preferably biodegradable and bioresorbable.

### Scaffolds

The tissue guidance devices of the present invention comprise at least one scaffold having a first end and a second end, and one or more lumen running through the scaffold from the first end to the second end. The scaffolds have a length measured from the first end to the second end, which is typically between about 1 mm and 50 mm. Preferably, the scaffold length is between about 1 mm and about 20 mm. Generally, the length of the scaffolds is chosen to match that of the tissue defect being repaired. The tissue guidance conduits of the present invention may be used to repair nerve gaps up to about 50 mm in length.

The scaffolds of the tissue guidance conduits of the present invention comprise one or more lumens and are formed of soft materials that are flexible, biocompatible, and preferably biodegradable and/or capable of being bioresorbed. In preferred embodiments, the scaffolds are formed of a hydrogel polymer that is preferably a charged hydrogel polymer. The scaffolds of the present invention may be formed with a biomimetic design that preferably closely matches the architecture of the tissue (such as one or more nerves) whose defect is to be repaired. When used for nerve regeneration, the biomimetic architecture of the scaffolds of the present invention has the advantage, amongst others, of promoting axon regeneration. The biomimetic structure of a scaffold may include, but is not limited to, one or more of varying the number, diameter and cross-section of the lumen(s), separating the lumen(s) into separate lumens to mimic nerve bundles (if the scaffold is used with nerves), varying the cross-sectional shape of the scaffold and/or lumen(s), changing the cross-sectional shape and/or area of the scaffold and/or one or more lumens along the length of the scaffold, or furcation of the scaffold into one or more branches along its length. Additionally, the cross-section and diameter of the scaffold may be chosen to promote axon regeneration.

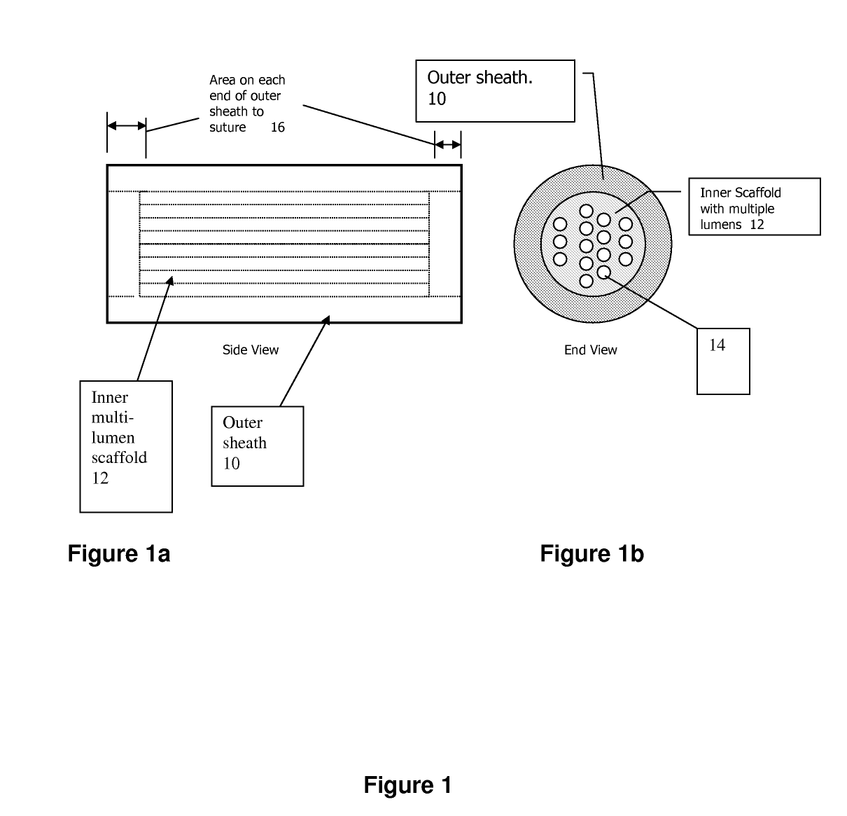

The scaffolds of the present invention are preferably either round or oval in cross-section, although any other suitable cross-section may be used. Examples of scaffolds with round or oval cross-sections are illustrated, for example, in FIGS. 1, 2 and 3. The diameter of the scaffold is typically in the range of about 0.5 mm to about 10.0 mm. Preferably, the diameter of the scaffold is in the range of about 2.0 mm to about 10.0 mm. Most preferred are scaffolds with a diameter in the range of about 1.0 mm to about 10.0 mm. In the above ranges, when the cross-section is not round (for example, oval) the diameter refers to the shortest dimension of the cross-section. In a preferred embodiment, the scaffold has an oval cross-section. When the scaffold has an oval cross-section the diameter is preferably in the range of about 0.5 mm by about 1.0 mm to about 7.0 mm by about 10.0 mm.

The diameter of the tissue regeneration device is a function of the tissue. Preferable diameters preferably match the tissue to be repaired. Some tissues have an oval cross-section, while others have a different (such as circular) cross-section. Preferable cross sections would be those with multiple lumens and possibly multi-lumen bundles that are separated by a septum.

The cross-sectional shape and/or diameter of a scaffold and/or one or more lumens may vary from the first end to the second end. For example, a scaffold and/or lumen cross-sectional shape or area may be greater at the first end of the scaffold than at the second end of the scaffold. In such a case, the cross-sectional shape or area may vary gradually along the length of the scaffold or may vary in a series of one or more steps along the length of the scaffold. For example, the scaffold cross-sectional shape can gradually change from round at the first end to oval at the second over its length or from oval at the first end to round at the second end.

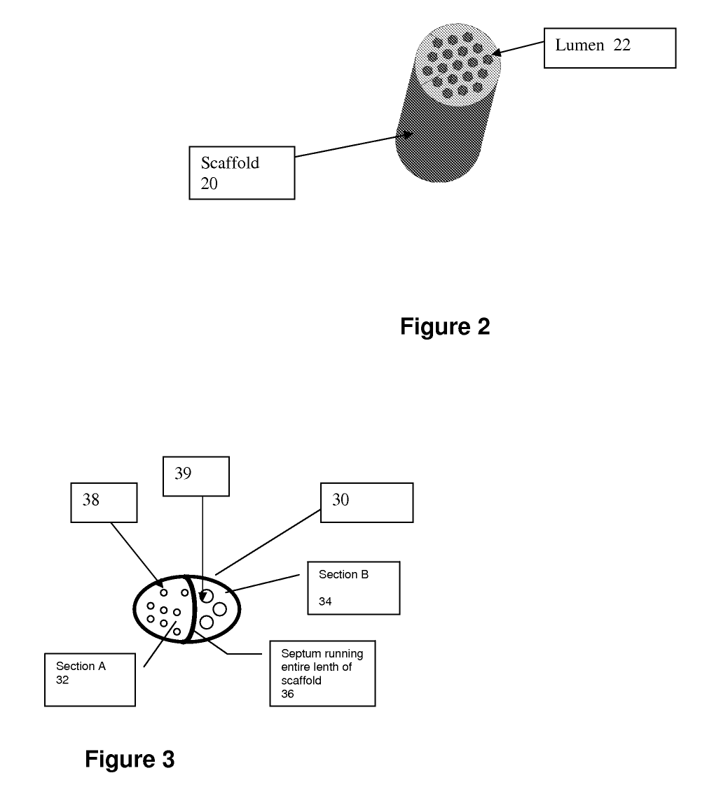

In one embodiment, the scaffold comprises a single section comprising one or more lumen, as illustrated, for example, in FIG. 2 wherein the scaffold 20 has lumen 22 running through it. In other designs the scaffold comprises two or more sections, for example as illustrated in FIG. 3. In FIG. 3 the scaffold 30 cross-section is oval and is divided into two sections 32 and 34. The sections are separated by a septum 36. The septum separates sections 32 and 34 from the first end to the second end of the scaffold. Each section comprises a bundle of one or more lumen 38 and 39. In this embodiment, the septum is formed of the same material as that of the scaffold, but it could be formed of another material.

Further, the scaffold can comprise more than two sections. For example, the scaffold may comprise three or more sections, four or more sections, five or more sections, or six or more sections. Each section may comprise one or more lumens and each section may have the same or a different number of lumens. Further, the cross-sectional area of each lumen in the same section and between sections may be the same or different. For example, as shown in FIG. 3, the lumen bundle of section 32 has a greater number of smaller diameter lumen 38 than the lumen bundle of section 34 with larger diameter lumen 39. In other embodiments, a lumen bundle may comprise one or more lumen with one or more different cross-sectional shapes and/or cross-sectional areas to other lumen in the bundle. In one embodiment, the first section of the scaffold comprises three lumen, and the second section comprises five lumen.

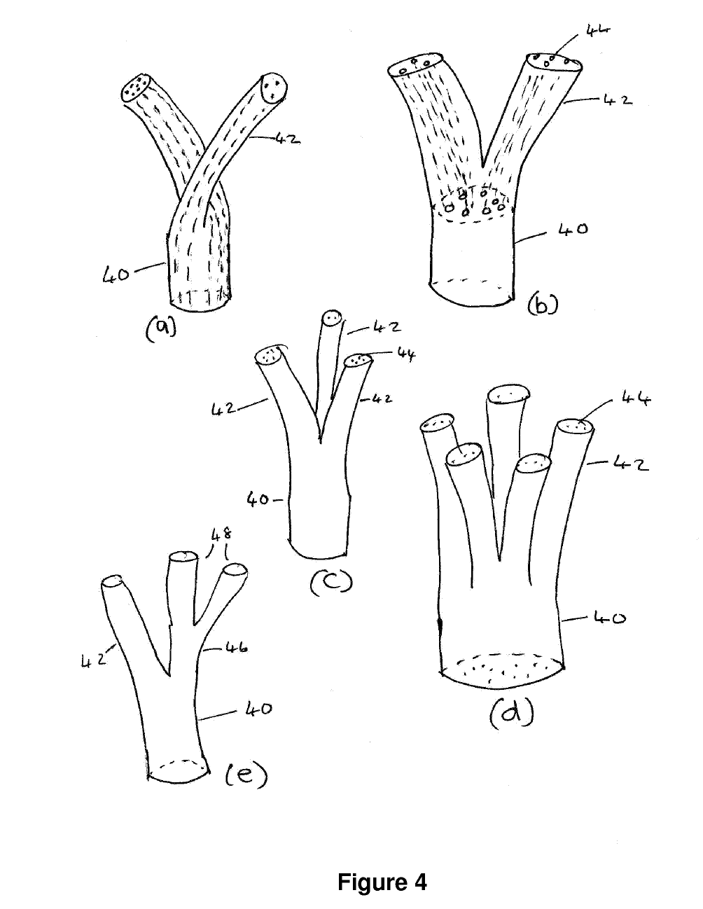

In other embodiments of the nerve guidance conduits of the present invention, the scaffold furcates into separate branches long the length of the scaffold. Some examples of branching scaffolds according to the invention are illustrated in FIGS. 4(a) to (e). In one embodiment, the scaffold bifurcates into two or more branches. In another embodiment, the scaffold trifurcates into three or more branches. Alternately, the scaffold furcates into four or more branches, five or more branches, or eight or more branches. In other nerve tube designs, the scaffold may branch more than once along its length as illustrated, for example, in FIG. 4(e). In FIG. 4(e) scaffold 40 furcates into branches 42 and 46, and branch 46 further furcates into branches 48. Further, in the branching designs a scaffold branch may change cross-section as it branches compared to that of the scaffold trunk or, alternately, change cross-section along the length of a branch.

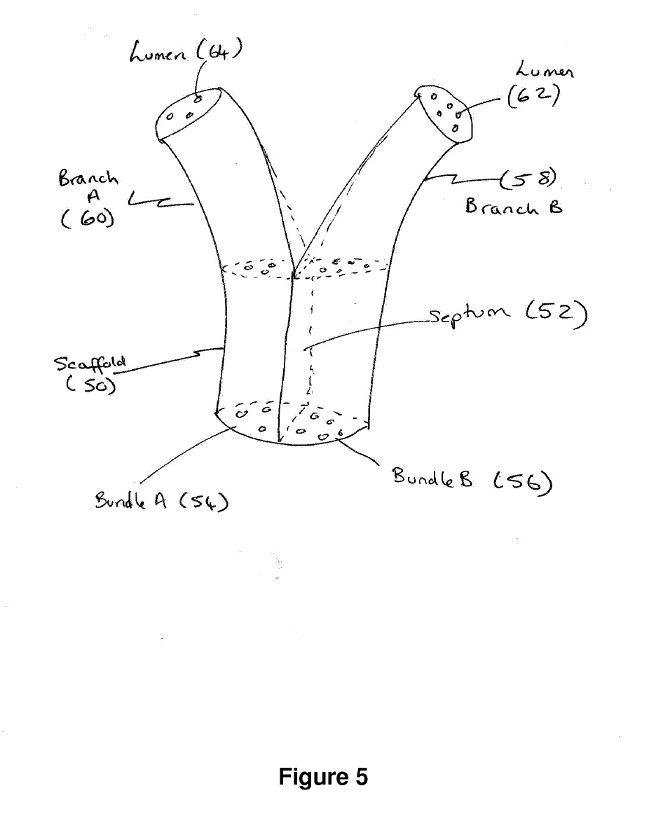

When scaffolds furcate into two or more branches the lumen are divided amongst the branches. Generally, each branch of the scaffold will comprise at least one or more lumen, and the lumen may or may not divide equally among the branches. FIG. 4(b) illustrates one example of a furcating scaffold 40 and the lumen 44 dividing amongst the branches 42. In a scaffold comprising two or more section separated by a septum, the scaffold may furcate such that each section forms a branch. This is illustrated, for example, in FIG. 5, where scaffold 50 has two sections 54 and 56 separated by septum 52. Along the length of the scaffold the sections split to form branches 58 and 60, each branch containing lumen bundles 62 and 64, respectively.

It is believed that branching scaffolds more closely mimic the architecture of biological systems leading to better results for axon regeneration. For example, by limiting axonal dispersion and by improving targeting of regenerating axons to the correct muscle. In one embodiment, the branching scaffold mimics that of the sciatic nerve.

### Lumen

The scaffolds of the present invention comprise one or more lumen running though the scaffold from the first end to the second end. In a preferred embodiment, the scaffold is a multi-lumen scaffold. In one embodiment, the multi-lumen scaffold comprises at least two or more lumen. Typically, the multi-lumen scaffold comprises three or more lumens, alternatively four or more lumens, five or more lumens, six or more lumens, seven or more lumens, eight or more lumens, nine or more lumens, ten or more lumens, eleven or more lumens, twelve or more lumens, thirteen or more lumens, fourteen or more lumens, fifteen or more lumen, sixteen or more lumens, seventeen or more lumens, eighteen or more lumens, or nineteen or more lumens.

The cross-sectional shape and/or area of the one or more lumen of the scaffold may vary considerably. For example, the lumen cross-section may be circular, hexagonal, octagonal, or oval, or alternatively have any other shape suitable for promoting axon regeneration (if used for nerve regeneration). In one embodiment, the lumen cross-section is circular. FIG. 1(b) illustrates a scaffold design with 15 lumen 14 of a circular cross-section. FIG. 2 illustrates an embodiment of a scaffold 20 with 19 lumens 22 of a hexagonal cross-section. In one embodiment, the cross-section of each of the plurality of lumen is the same. In another embodiment, the cross-sectional shape of at least one lumen in the scaffold is different from that of the cross-sectional shape of at least one other lumen in the scaffold. In one embodiment, the scaffold comprises lumens of at least two different cross-sectional shapes.

The cross-sectional area of the lumen may vary considerably. The lumen diameter can vary from about 20 μm to about 1000 μm. In some embodiments, the lumens have a diameter in the range of about 75 μm to about 600 μm. In certain embodiments, the lumens have a diameter in the range of about 200 μm to about 450 μm. In the above ranges, when the cross-section is not circular, for example oval or hexagonal, the diameter refers to the shortest dimension of the cross-section. In one embodiment the cross-sectional area of each of the one or more lumens is the same. In another embodiment, the cross-sectional area of at least one of the lumens is different from that of at least one of the other lumens in the scaffold. In alternate embodiments, the scaffold may comprise lumens of at least two different diameters. It is preferable that the cross-sectional shape and diameter of the lumen is chosen to match that of the tissue whose defect is to be repaired. For example, the cross-sectional area and diameter of the lumen may be chosen to match that of the sciatic nerve.

The cross-section and/or cross-sectional area of a lumen may also vary from first end of the scaffold to the second end of the scaffold. For example, a lumen diameter may be greater at the first end of the scaffold than at the second end of the scaffold. In such a case, the diameter may gradually change along the length of the scaffold or may change in a series of one or more steps along the length of the scaffold. In another example, the lumen cross-sectional shape may be hexagonal at the first end and gradually change to circular at the second end. In one embodiment of the tissue regeneration devices of the present invention, one or more of the lumen of a scaffold change cross-sectional shape and/or diameter along the length of the scaffold. In another embodiment, all the lumen of the scaffold change cross-sectional shape and/or diameter along the length of the scaffold.

The number of lumen at the first end of the scaffold may be the same or different to the number of lumen at the second end of the scaffold. For example, a lumen may branch along the length of the scaffold into two or more lumens. In one embodiment the number of lumen at the first end of the scaffold is less than the number of lumen at the second end of the scaffold.

### Materials for Forming Scaffolds

The scaffolds of the present invention are formed from a soft biocompatible material having suitable mechanical and physical properties. Preferred materials are biocompatible polymers that are also biodegradable and/or bioresorbable. In one embodiment, the scaffold is bioresorbable. In another embodiment, the scaffold is biodegradable. Materials used to form the scaffold are preferably permeable or porous to water soluble nutrients, small molecules and gases essential to axon regeneration. Materials must also maintain their structural integrity in-vivo for a period long enough to allow axon regeneration (if used to regenerate nerves) before biodegradation and/or bioresorbtion. Preferably, the scaffolds maintain their structural integrity for a period of at least 3 months or more after implantation. More preferred, are periods of 3 to 9 months. One method to regulate the rate of biodegradation is by controlling the amount of cross-linking of the polymer forming the scaffold.

Preferred materials for making the scaffolds of the present invention, and other applications described herein, are polymer hydrogels, although other suitable biocompatible polymers may also be used. A material such as a hydrogel has several advantages over competing alternatives. The matrix adhesion ligand concentration, charge density and porosity can be systematically altered by the organic synthesis design. By manipulation of a fixed charge or ligand density within hydrogel cellular attachment and function can be altered and designed for a particular application (see, for example, E. Alsberge et al., J. Dent. Res. 2001; 80, 2025-9 and A. E. English et al., Polymer 1998; 39, 5893, the disclosures of which that are not inconsistent with the disclosure herein are incorporated by reference).

Particularly preferred for forming the scaffolds of the present invention are charged hydrogel polymers. The charged polymers hydrogels may be either positively or negatively charged, but positively charged hydrogel polymers are most preferred. In one embodiment, the scaffold comprises a positively charged hydrogel. Positively charged hydrogel polymers offer certain advantages over non-charged polymers, including promoting cellular growth and/or enhanced biocompatibility. While not being bound by any particular theory, it is believed that the charge of the hydrogel polymers promotes cellular growth. In particular, charged hydrogel polymers are particularly effective at promoting nerve cell growth and nerve regeneration. It has been observed that while cellular growth occurs readily on charged hydrogel polymer supports, the same growth habits are not observed for supports formed from uncharged polymers, and that for some polymers no nerve cell growth is observed at all. Positively charge hydrogels also have the advantage of being able to promote growth of a myelin sheath on nerve cells, as well as promoting the growth of both peripheral and central nerve cells.

Examples of biocompatible polymers for forming hydrogel polymers and charged hydrogel polymers for the scaffolds of the present invention include, but are not limited to, one or more polymers selected from the group consisting of oligo-(polyetheneglycol)fumarate hydrogels (OPF), polycaprolactone fumarate (PCLF), polycaprolactone fumarate/polypropylene fumarate copolymer (PCL-PPF), polyethylene glycol fumarate (PEGF), PEGF-PCLF, PEG-PPF, hydrophilic/hydrophobic PEGF-PCLF, hydrophilic/hydrophobic PEGF-PPF, and co-polymers of each. Examples of OPF polymers include those described in International Patent Application No. PCT/US2006/010629 to Dadsetan et al., the disclosure of which that is not inconsistent with the disclosure herein is incorporated by reference.

In preferred embodiments, the charged polymer hydrogels comprise oligo-(polyethylene glycol)fumarate (OPF) hydrogels. The oligo-(polyethylene glycol)fumarate (OPF) hydrogels of the present invention have significantly higher mechanical properties than other hydrogels, although the scaffolds can be formed of any material with suitable properties. One advantage of OPF hydrogels, compared, for example, to polymers such as PEGF, is that they have greater biocompatibility with cells in living systems. In particular they may be formed using crosslinking agents and/or photoinitiators that are less toxic to living cells. Hydrogel polymers also have properties that allow them to be readily formed into biomimetic structures by the methods described herein.

The positively charged hydrogels of the present invention may be synthesized by the copolymerization of a positively charged monomer with a polymer hydrogel. Any positively charged monomer may be used in the embodiments of the present invention. In one embodiment, the positively charged monomer is [2-(methacryloyoxy)ethyl]-trimethylammonium chloride (MAETAC). In one embodiment of the present invention, oligo (polyethylene glycol)fumarate (OPF) hydrogel is copolymerized with [2-(methacryloyloxy)ethyl]-trimethylammonium chloride (MAETAC) to produce a positively charged hydrogel. This charged hydrogel can be cross-linked by either light or redox-initiated systems and fabricated into tubes, sheets, sponges, microspheres and other forms. Controlling the degree of cross-linking can be used to control the structural stability of the polymers of the nerve guidance conduits and other applications of the present invention. Further, by controlling the degree of charge or density of charge of the hydrogel, by varying for example the ratio of the charge monomer to the polymer, the cellular growth properties of the charged hydrogel may be modified. Similar approaches for introducing positively charged monomers into other polymers and hydrogels may also be used to practice the present invention.

Generally, the average molecular weight of the hydrogel, or other polymer, is chosen to be in the range of about 1,000 Daltons (Da) to about 20,000 Da or any suitable range therein. More preferably, the molecular weight is in between about 4,000 to about 15,000 Da, alternatively between about 5,000 Da to about 13,000 Da or between about 6,500 Da to about 12,500 Da. In one embodiment, the OPF hydrogels are used with a molecular weight of about 1,000 Da to provide for permeability.

The surfaces of the scaffolds and/or lumen may also be modified or coated to increase compatibility with regenerating tissue, for example to regenerate axons and further promote nerve regeneration. Surfaces may be modified, for example, by attaching bioactive agents as discussed herein, or by treatments (such as, for example, etching the surface by incubation in 80% ethanol) to enhance the attachment of neurons, axons, Schwann cells, stem cells, or bioactive agents to the surface. Some agents may also be formed into the surfaces of the scaffolds and/or lumen.

The scaffolds of the present invention may additionally comprise bio-active agents, such as growth factors. As used herein the term “active agent” refers to any substance that is capable of providing a therapeutic, prophylactic or other biological effect within a patient. An active agent can also be a diagnostic agent. An active agent can be a drug. Active agents include synthetic inorganic and organic compounds, proteins and peptides, polysaccharides and other sugars, lipids, and DNA and RNA nucleic acid sequences having therapeutic, prophylactic or diagnostic activities. Examples of bio-active agents include, but are not limited to, nerve growth factors and cell growth factors. Examples of nerve growth factors include, but are not limited to, stem cells, adult stem cells, Schwann cells, fibronectin, laminin, neural cell adhesion molecules (N-CAM), and active peptide derived from N-CAM. Bio-active agents are preferably incorporated on the surface of the lumen wall, but may also be incorporated into the scaffold body, on the outer surfaces of the scaffold, incorporated the material forming the outer sheath, and on the inner or outer surface of the outer sheath.

### Methods of Making Scaffolds

The scaffolds of the present invention may be constructed using techniques such as, for example, vacuum molding, by extrusion techniques, or by layered based fabrication technology. The scaffolds are preferably constructed using layered-based fabrication (LBF) technology, such as stereo-lithography or laser stereo-photolithography. One advantage of these techniques is that they allow scaffolds to be built at the nanometer and micrometer level using computer aided design and laser stereo/photo lithography to cross-link photopolymerizable polymers and allow for the construction of scaffolds and lumens of a chosen geometry. In particular, laser stereo/photo lithography provides for the production of complex biomimetic structures. Layer based fabrication technology is especially preferred when the scaffolds are formed of OPF-hydrogels. In one embodiment, UV-cured LBF technology is used to form the individual cross sections of the scaffolds of the present invention. A photopolymer is exposed to a UV or visible light wavelength energy source. A physical, or digital mask that defines either the positive or negative geometry of the scaffold is used to determine the portion of the photopolymer will be solidified. This initiates the polymerization and a thin layer (cross section) is solidified. New material (polymer) is applied to the previously solidified layer, and it is again exposed to UV or visible light, or other suitable method, to polymerize, using the aforementioned mask to determine the cross section. Each cross section represents a “slice” of the entire device. In an embodiment, a device useful for making scaffolding is an SLA Viper Machine (from 3D Systems Corporation, South Carolina). Another device that might be useful for making scaffolding is the Perfactory (from Envisiontec, Michigan).

Another advantage of using LBF is that ridges or other structures may be incorporated into the lumen geometry that can enhance cell attachment to the side of the lumen walls. In addition, layer-based fabrication methods have the advantage of being able to form scaffold structures that biomimetic biological tissue, such as nerve structure.

### Outer Sheath

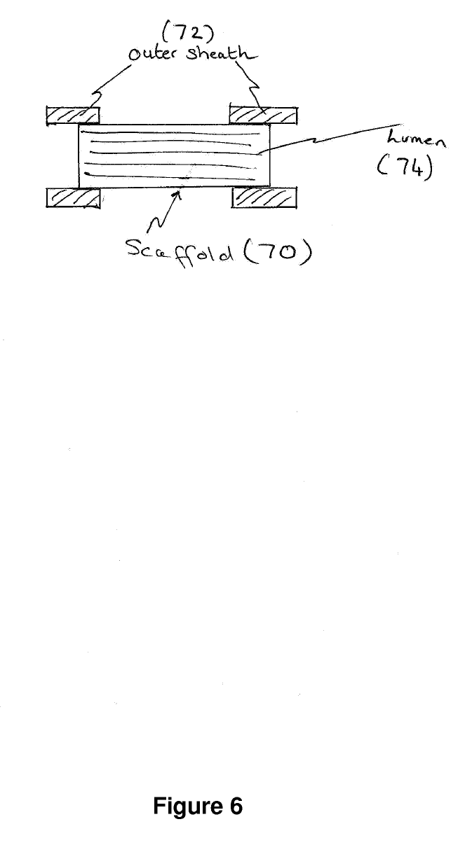

Another aspect of the present invention involves the construction of a tissue guidance conduit comprising a scaffold, as described herein, and an outer sheath. The outer sheath surrounds all or part of the outer surface of the scaffold. FIGS. 1(a) and (b) illustrates one embodiment wherein the outer sheath 10 completely surrounds the scaffold 12. In alternate embodiments, such as shown in FIG. 6, the outer sheath 72 may surround a portion of the scaffold 70 at each end. After forming a scaffold, as described herein, the scaffold is pushed or slipped into the outer sheath to form a tissue guidance device according to the invention. In one embodiment, the scaffold, comprising, for example, a OPF hydrogel, is slipped or pushed into the outer sheath using any suitable method.

The outer sheath is formed of a material with mechanical properties to make it flexible and stronger than the scaffold, and which allows the outer sheath to be securely sutured in place with greater resistance to tearing than the scaffold. The outer sheath is chosen to be biocompatible, and is preferably biodegradable, and/or bioresorbable, as described above for the inner scaffold. In one embodiment, the outer sheath has a tear strength that is greater than the tear strength of the inner scaffold. The tensile/tear strength of the outer sheath may be similar to that of the nerve to be regenerated.

The outer sheath is preferably formed of a polymer, but other biocompatible materials may also be used. Examples of polymers for forming the outer sheath include, but are not limited to, PCL (polycaprolactone), polycaprolactone fumarate (PCLF), polypropylene fumarate (PPF), polycaprolactone fumarate/polypropylene fumarate copolymer, polyethylene glycol fumarate (PEGF), PEGF-PCLF, PEG-PPF, hydrophilic/hydrophobic PEGF-PCLF, hydrophilic/hydrophobic PEGF-PPF, and copolymers of each. In one embodiment, the outer sheath is formed of PCL (polycaprolactone). The outer sheath preferably has a wall thickness of between about 50 microns to about 250 microns. In some embodiments, the outer sheath wall thickness is from about 100 microns to about 200 microns.

The outer sheath has a first end and a second end, and preferably a portion at each end for suturing the tissue guidance conduit to the nerve ends. In a preferred embodiment, a portion at the first end of the outer sheath extends beyond the first end of the scaffold and a portion at the second end of the outer sheath extends beyond the second end of the scaffold, as illustrated in FIG. 1 where area 16 of the outer sheath 10 extends beyond the end of the scaffold 12. Preferably each end portion of the outer sheath extends about 0.5 mm to about 5.0 mm beyond the end of the scaffold with which it is juxtaposed, and more preferably about 1.0 mm to about 3.0 mm beyond such end of the scaffold. One or both end portions of the outer sheath may further comprise a raised lip (a portion at the end of the outer sheath that is thickened) to further aid the suturing of the outer sheath to the nerve ends.

The outer sheath may optionally comprise one or more perforations formed in it in any suitable manner. In one embodiment, the perforations may be cut into the outer sheath by laser milling. Perforations in the outer sheath can enhance mass transport properties of the tissue guidance conduit and promote healthy cell growth by allowing the diffusion of molecules and solutes though the outer sheath wall to the regenerating tissue in the scaffold lumen. Perforations are preferably a pattern of micro-sized holes that are cut in the outer sheath prior to inserting the assembled inner scaffold. The size and pattern of the perforations, and the number perforations per area can vary widely. Generally, the number of perforation per mm² of outer sheath is between about 1 perforations per mm² and about 100 perforations per mm², more preferably between about 1 perforations per mm² and about 20 perforations per mm², and most preferably between about 1 perforations per mm² and about 10 perforations per mm². The number of perforations per mm² can be uniform along the length of the outer sheath or, alternately, can vary along its length either in one or more increasing or decreasing steps. The number of perforations per mm² may also gradually increase or decrease from one end of the outer sheath to the other end of the outer sheath. In one embodiment, a greater number of perforations per mm² is in the center of the outer sheath than in the ends. Similarly, the one or more perforations may have a uniform size or may vary in size along the length of the outer sheath.

When perforations vary in size, the size can change uniformly along the length, change randomly along the length, or change in a series of size increasing or decreasing steps. For example, the size of the perforation may be greater in the central section than in the end sections of the outer sheath. Alternately, the size may gradually increase from one end to the central section and then decrease gradually to the second end. In one embodiment, at least one of the one or more perforations is of a different size from other perforations in the outer sheath. Generally, the size of each of the one or more perforations is between about 10 μm to about 250 μm, or any range therein. More preferably, the sizes of the perforations are between of about 10 μm to about 500 μm, alternately in the range of about 10 μm to about 100 μm or in the range of about 10 μm to about 50 μm. In certain embodiments, when perforations are cut in the outer sheath, a portion at each end is left without perforations to allow the outer sheath to be sutured in place. In one embodiment, the perforations may be cut into the outer sheath by laser milling.

### Other Applications of Charged Hydrogels

Other applications for the positively charged hydrogels of the present invention described herein include, but not limited to, cartilage, bone, muscle and soft tissue regeneration and/or repair, treatment of burns by allowing burn victims to re-grow skin, and other tissue engineering applications. Charged hydrogel polymers are also useful for applications where a biocompatible medium or support are used to provide controlled drug delivery, DNA delivery and cell transplantation. The charged hydrogel will be manufactured in any suitable manner and shape for an application in which it is to be used. For example, it could be manufactured as a thin sheet to help regenerate burned tissue (such as skin), or as a tape to wrap torn tendons or broken bones. The hydrogel could also be placed in-situ as a liquid to conform it to a particular area of the body with tissue to be regenerated and then cured in-situ. Active agents may be applied to the hydrogel for any of these applications.

As one example, hydrogels with a positively charged group are useful for controlled gene delivery. Current strategies to enhance nonviral gene delivery involve the complexation of DNA with cationic polymers or lipids. Cationic polymers or lipids can self-assemble with DNA to form particles that are capable of being endocytosed by cells (see, for example, T. Segura, et al. J. Control. Release 2003, 93, 69-84, and W. C. Tseng, J. Biol. Chem. 1997, 41, 25641-25674, the disclosures of which that are not inconsistent with the disclosure herein are incorporated by reference). In general, DNA delivery from biomaterials can be categorized into two fundamental approaches: sustained release and immobilization. Sustained-release systems are designed to maintain elevated concentrations locally by supplying DNA to balance the loss by degradation or clearance. Alternatively, DNA can be immobilized within a biomaterial scaffold. Synthetic systems based on the immobilization of nonviral DNA complexes have a guiding principle that the substrate must be designed to maintain the DNA locally, yet allow for cellular internalization. Therefore, addition of a positive charge to the hydrogel facilitates the transfection of the cells by increasing the concentration of the DNA in the cellular microenvironment. Similarly, incorporation of drugs or bioactive agents in positively charged hydrogels can facilitate the delivery of active agents to target sites.

### Synthesis and Characterization of Charged Hydrogel Polymers

Oligo-(polyethylene glycol)fumarate (OPF) with an weight average molecular weight of 16,246±3710 Da was synthesized using polyethylene glycol (PEG) with the initial molecular weight of 10,000 Da according to the method described in S. Jo et al., Biomacromolecules, 2001, 2(1), 255-61, the disclosure of which that are not inconsistent with the disclosure herein is incorporated by reference.

Positively charged modified OPF hydrogels were synthesized by dissolving the OPF macromer to a final concentration of 33% (w/w, which means “weight percentage”) in deionized water containing 0.05% (w/w) of a photoinitiator (Irgacure 2959, Ciba-Specialty Chemicals) and 0.33% (w/w) N-vinyl pyrrolidinone (NVP). To produce positively charged hydrogels, MAETAC (75%, Aldrich) at the concentrations of 0.1, 0.2 and 1 M was added to the solution. The OPF/MAETAC mixture was pipetted between glass slides with a 1 mm spacer and polymerized using UV light (365 nm) at an intensity of ˜8 mW/cm² (Blak-Ray Model 100AP) for 30 min.

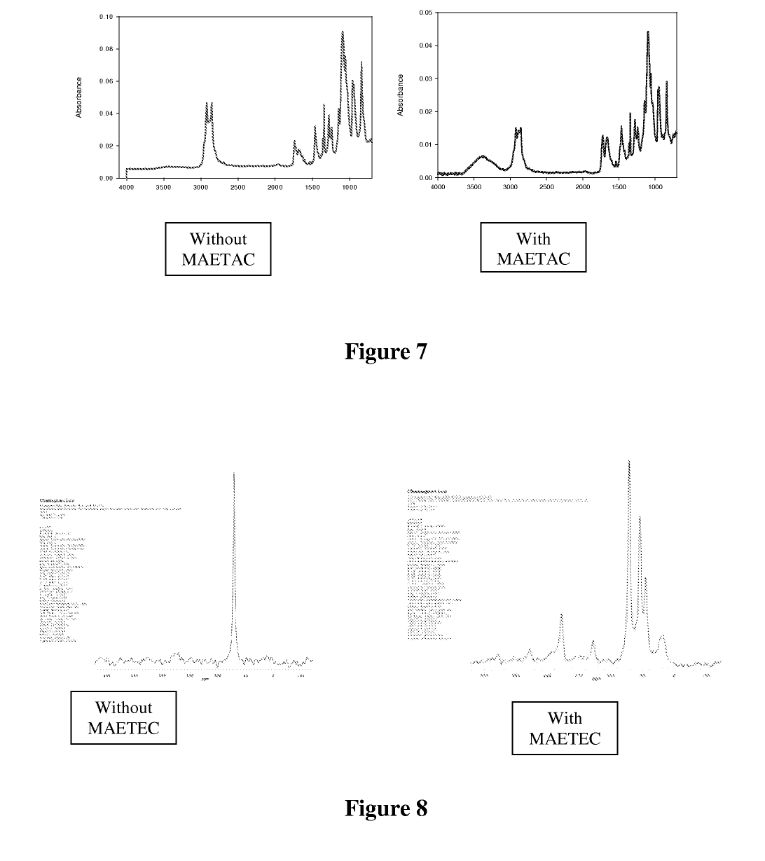

The presence of MAETAC in the charged hydrogel polymer was characterized by ATR-FTIR and NMP spectroscopy. ATR-FTIR confirmed the presence of MAETAC on the crosslinked hydrogels as shown in FIG. 7. Solid state C13 NMR showed the presence of new peaks on the hydrogels corresponding to ammonium salt as shown in FIG. 8.

After crosslinking, the hydrogels were cut into disks with a diameter of 10 mm and swollen in phosphate buffered saline (PBS) and deionized water for 24 hours at 37° C. Swollen samples were blotted, dried, and weighed (Ws), and then dried in reduced pressure and weighed again (Wd). Swelling ratio of hydrogels was calculated using following equation:

Swelling ratio=Ws−Wd/Wd

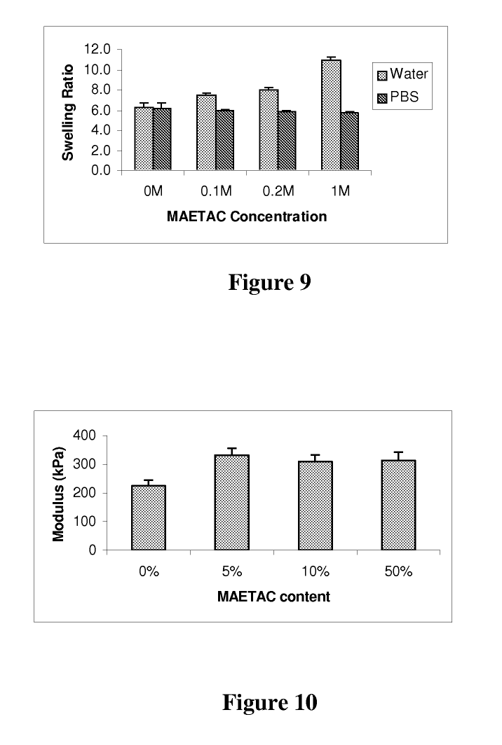

FIG. 9 shows swelling of positively charged hydrogels in PBS and water. Swelling ratios of hydrogels increased in water significantly with the increase in concentration of MAETAC, while they remained constant in PBS, indicative of the ionic nature of the modified hydrogels.

The mechanical properties of the charged hydrogels were tests as follows. Compressive modulus of the various swollen hydrogels was determined using a dynamic mechanical analyzer (DMA-2980, TA Instruments) at a rate of 4 N/min. The modulus was determined as the slope of the stress versus strain curve at low strains. FIG. 10 shows that compressive modulus of OPF hydrogels increased with the addition of MAETAC.

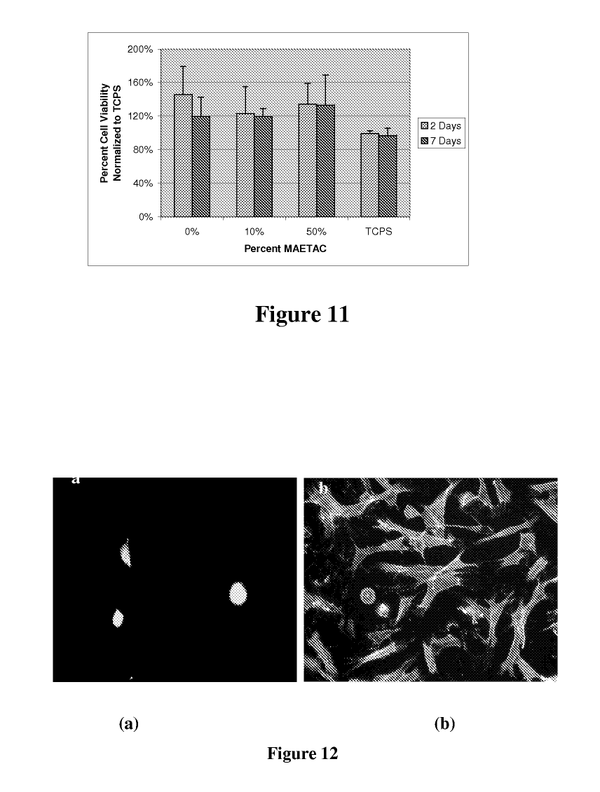

Cell viability was tested using the MTS viability test. The MTS cell proliferation assay is a calorimetric method to identify the cytotoxic potential of a test item. The assay measures the formation of a soluble formazan product which is directly proportional to the number of live cells in culture. MTS viability test showed high viability for marrow stromal cells cultured in the presence of charged hydrogels after 2 and 7 days. FIG. 11 shows the results of cell viability tests as a percentage of MAETAC.

In cell attachment tests positively charged OPF hydrogels supported greater cell attachment as compared to unmodified hydrogels. Cell spreading correlated to cytoskeleton development and differentiation of marrow stromal cells (MSC) was characterized using actin filament fluorescence staining. FIG. 12 shows MSCs attached to (a) unmodified and (b) charged modified OPF hydrogels (0.2 M MAETAC). As shown in FIG. 11, MSCs attached and spread more readily on the surface of positively charged hydrogels than unmodified hydrogels after 24 hours.

While particular embodiments of the present invention have been described, changes and modifications can be made without departing from the spirit and scope of the teachings and embodiments of this invention. Such teachings are provided in the way of example only, and are not intended to limit the scope of the invention, which is set forth in the appended claims and legal equivalents thereof.

What is claimed is:

1. A tissue regeneration device, the device comprising a scaffold having a first end and a second end, and one or more lumens running through the scaffold from the first end to the second end.

2. The tissue regeneration device of claim 1 wherein the scaffold comprises a charged polymer.

3. The tissue regeneration device of claim 3 wherein the scaffold comprises a charged hydrogel polymer.

4. The tissue regeneration device of claim 1 wherein the scaffold has a biomimetic structure.

5. The tissue regeneration device of claim 1 wherein at least a portion of the scaffold is positioned in an outer sheath.

6. The tissue regeneration device of claim 5 wherein the scaffold is positioned entirely in the outer sheath.

7. The device of claim 5 wherein the outer sheath is permeable.

8. The device of claim 1 wherein the structure is configured for the regeneration of nerves.

9. The device of claim 3 wherein the hydrogel polymer is a positively charged hydrogel.

10. The device of claim 6 wherein the scaffold has an outer surface and the outer sheath surrounds the entire outer surface of the scaffold.

11. The device of claim 10, wherein the outer sheath surrounds a portion of the outer surface of the scaffold.

12. The device of claim 11, wherein the portion of the scaffold surrounded by the outer sheath includes at least the first end of the scaffold and the second of the scaffold.

13. The device of claim 10, wherein the outer sheath comprises a first end, a second end, and a portion at each of the ends to suture the device to body tissue.

14. The device of claim 13, wherein the outer sheath is configured to be sutured to nerve ends.

15. The device of claim 6, wherein the first end of the outer sheath extends beyond the first end of the scaffold, and the second end of the outer sheath extends beyond the second end of the scaffold.

16. The device of claim 13, wherein the first end and second end of the outer sheath each comprise a raised lip.

17. The device of claim 16, wherein the raised lip on each of the ends is used to suture the device to nerve endings.

18. The device of claim 5, wherein the tear strength of the outer sheath is greater than the tear strength of the scaffold.

19. The device of claim 5, wherein the thickness the outer sheath is between 50 and 250 microns.

20. The device of claim 5, wherein the outer sheath comprises one or more perforations.

21. The device of claim 20, wherein the size of each of the perforations is in the range of about 10 μm to about 250 μm.

22. The device of claim 21, wherein the number of perforations is in the range of about 1 to about 100 perforations per mm².

23. The device of claim 21, wherein the number of perforations per unit area of the outer sheath is uniform along the length of the outer sheath.

24. The device of claim 21, wherein the number of perforations per unit area of the outer sheath varies along the length of the outer sheath.

25. The device of claim 21, wherein the number of perforations per unit area of the outer sheath is greater in the central portion of the outer sheath than in portions at each end of the outer sheath.

26. The device of claim 7, wherein the scaffold and outer sheath allow for the diffusion of molecules and solutes therethough.

27. The device of claim 1, wherein the device is biodegradable and/or bioresorbable.

28. The device of claim 5, wherein the device is biodegradable and/or bioresorbable.

29. The device of claim 3, wherein the positively charged polymer is formed by the copolymerization of a positively charged monomer with one or more polymers or monomers.

30. The device of claim 29, wherein the positively charged monomer is [2-(methacryloyoxy)ethyl]-trimethylammonium chloride (MAETAC).

31. The device of claim 29, wherein the positively charged polymer is cured by UV or visible light or redox-initiated cross-linking of the positively charged monomer to the polymer.

32. The device of claim 3, wherein the positively charged hydrogel is formed by the copolymerization of [2-(methacryloyoxy)ethyl]-trimethylammonium chloride (MAETAC) and oligo-(polyetheneglycol)fumarate hydrogel (OPF).

33. The device of claim 1, wherein the scaffold comprises one or more polymers selected from the group consisting of oligo-(polyetheneglycol)fumarate hydrogel (OPF), polycaprolactone fumarate (PCLF), polycaprolactone fumarate/polypropylene fumarate copolymer (PCL-PPF), polyethylene glycol fumarate (PEGF), PEGF-PCLF, PEG-PPF, hydrophilic/hydrophobic PEGF-PCLF, hydrophilic/hydrophobic PEGF-PPF, and co-polymers of each.

34. The device of claim 5, wherein the outer sheath is formed of a material selected from one or more of polycaprolactone (PCL), polycaprolactone fumarate (PCLF), polypropylene fumarate (PPF), polycaprolactone fumarate/polypropylene fumarate copolymer, polyethylene glycol fumarate (PEGF), PEGF-PCLF, PEG-PPF, hydrophilic/hydrophobic PEGF-PCLF, hydrophilic/hydrophobic PEGF-PPF, and copolymers of each.

35. The device of claim 1 that comprises 2 or more lumens.

36. The device of claim 1 that comprises 3 or more lumens.

37. The device of claim 1 that comprises 5 or more lumens.

38. The device of claim 1 that comprises 7 or more lumens.

39. The device of claim 1 that comprises 19 or more lumens.

40. The device of claim 1, wherein the cross-sectional area of each of the one or more lumens is the same.

41. The device of claim 1 that has a plurality of lumens and the cross-sectional area of at least one of the lumens is different than the cross-sectional area of at least one of the other lumens.

42. The device of claim 1 that has a plurality of lumens and the cross-sectional area of at least one of the lumens at the first end of the scaffold is different from the cross-sectional area of the same lumen at the second end of the scaffold.

43. The device of claim 1 that has a plurality of lumens and the cross-sectional area of at least one of the one or more lumens is greater at the first end of the scaffold than at the second end of the scaffold.

44. The device of claim 1, wherein the scaffold has a round or an oval cross-section.

45. The device of claim 1, wherein the scaffold comprises two or more sections and each section comprises one or more lumens.

46. The device of claim 45, wherein the one or more sections of the scaffold are separated by a septum running from the first end of the scaffold to the second end of the scaffold.

47. The device of claim 45, wherein the cross-sectional area of at least one of the lumens in one section is different from the cross-sectional area of at least one of the lumens in the other sections.

48. The device of claim 45, wherein the number of lumens in each section is different.

49. The device of claim 45, wherein the number of lumens in each section is the same.

50. The device of claim 45, wherein the scaffold comprises a first section and a second section and the first section comprises three or more lumens and the second section comprises five or more lumens, and the number of lumens in the first section is less than the number of lumens in the second section.

51. The device of claim 1, wherein at least one of the one or more lumens branches along the length of the scaffold into two or more lumens, whereby the number of lumens at the first end of the scaffold is less than the number of lumens at the second end of the scaffold.

52. The device of claim 1, wherein the scaffold furcates along its length into two or more scaffold branches, such that each of the one or more branches comprises one or more lumens.

53. The device of claim 1 wherein each of the lumens has a round cross-section selected from one or more of the following shapes: oval, hexagonal, and octagonal.

54. The device of claim 1, wherein the diameter of each of the lumens is from about 20 μm to about 1000 μm.

55. The device of claim 1, wherein the diameter of the scaffold is at least about 100 μm.

56. The device of claim 1, wherein the diameter of the scaffold is from about 100 μm to about 4000 μm.

57. The device of claim 1, wherein the cross-section of the scaffold has dimensions of about 100 μm by 150 μm to about 1000 μm by 4000 μm.

58. The device of claim 1, wherein the device has a length from the first end of the scaffold to the second end of the scaffold of at least about 0.5 mm.

59. The device of claim 1, wherein the length of the scaffold is between about 0.5 mm and about 50 mm.

60. The device of claim 6, wherein the length of the outer sheath is greater than the length of the scaffold.

61. The device of claim 1, wherein the device is used in the repair of peripheral nerves.

62. The device of claim 1, wherein scaffold further comprises one or more bio-active agents.

63. The device of claim 62, wherein the bio-active agent is a nerve growth factor.

64. The device of claim 63, wherein the nerve growth factor is selected from one or more of the group consisting of stem cells, adult stem cells, Schwann cells, fibronectin, laminin, neural cell adhesion molecules (N-CAM), and active peptide derived from N-CAM.

65. A method for regenerating a nerve comprising placing a tissue regeneration device between two nerve ends, wherein the device comprises a scaffold having an outer surface, a first end and a second end, and one or more lumens extending from the first end to the second end, wherein the scaffold is formed of a charged polymer hydrogel.

66. The method of claim 65, wherein the maximum gap between the nerve ends is about 50 mm.

67. The method of claim 65, wherein the gap between the nerve ends is about 0.5 mm and 50 mm.

68. The method of claim 65, wherein the device further comprises an outer sheath surrounding at least part of the outer surface of the scaffold.

69. The method of claim 68, wherein the outer sheath is configured to be sutured to the nerve ends.

70. A method of making a nerve regeneration device comprising:
(a) forming a scaffold comprised of one or more lumens;
(b) forming a outer sheath;
(c) inserting at least a portion of the scaffold into the outer sheath.

71. The method of claim 70, wherein the scaffold comprises a charged hydrogel.

72. The method of claim 70, wherein the scaffold is formed by a method selected from the group consisting of layer-based fabrication technology, laser stereo-photolithography, extrusion techniques, vacuum molding, and combinations thereof.

73. The method of claim 72, wherein the scaffold is cured by photosensitive, UV or visible light-cured layer based fabrication technology.

74. The method of claim 70, wherein the scaffold is inserted into the outer sheath by human or mechanical means.

75. The method of claim 70, wherein the outer sheath has one or more perforations formed in it prior to inserting the scaffold into the outer sheath.

76. The method of claim 75, wherein the one or more perforations are formed by laser milling or by puncturing.

77. A method for promoting the healing/repair of body tissue, nerve cells, bone, cartilage or muscle, wherein the method comprises:
forming a charged substrate comprising a charged hydrogel polymer; and
applying the charged substrate to the region of body tissue, nerve, bone, cartilage or muscle to be repaired;
wherein the charged hydrogel polymer stimulates cellular growth thereby promoting repair and/or healing.

78. The method of claim 77, wherein the charged hydrogel polymer is a OPF charged hydrogel polymer.

79. The method of claim 77, wherein the substrate further comprises one or more bio-active agents.

80. The method of claim 77, wherein the hydrogel is positively charged.

81. The method of claim 80, wherein the charged polymer is formed by the copolymerization of a positively charged monomer with one or more hydrogel polymers.

82. The method of claim 81, wherein the positively charged monomer is [2-(methacryloyoxy)ethyl]-trimethylammonium chloride (MAETAC).

83. The method of claim 81, wherein the positively charged polymer is formed by uv or visible light or redox-initiated cross-linking of the positively charged monomer to the polymer.

84. The method of claim 82, wherein the positively charged hydrogel is formed by the copolymerization of [2-(methacryloyoxy)ethyl]-trimethylammonium chloride (MAETAC) and oligo-(polyetheneglycol)fumarate hydrogel (OPF).

85. A method for the controlled delivery of a bioactive agent to a target site in a body, wherein the method comprises:
forming a material comprising a charged hydrogel polymer;
incorporating a bioactive agent into the substrate; and
placing the material at the target site.

86. The method of claim 85, wherein the bioactive agent is one or more of a gene, a drug and cells.

87. A biocompatible and bioreabsorable material formed of a charged hydrogel and being configured to fit an area of a body wherein tissue is to be repaired, the material being in the form of a sheet, a tape or a liquid to be cured in-situ.

88. The device of claim 1 that has a plurality of lumens and the cross-sectional shape of at least one of the lumens is different from the cross-sectional shape of at least one of the other lumens.

89. The device of claim 1 that has a plurality of lumens and the cross-sectional shape of at least one of the lumens at the first end of the scaffold is different from the cross-sectional shape of the same lumen at the second end of the scaffold.
